(19) United States
(12) Patent Application Publication
(10) Pub. No.: US 2017/0216618 A1
(43) Pub. Date: Aug. 3, 2017

(54) PLAQUE TARGETED SUB-BLISTERING DOSIMETRY

(71) Applicant: PHOTOMEDEX, INC., Horsham, PA (US)

(72) Inventors: John KOO, San Francisco, CA (US); Maya DEBBANEH, San Francisco, CA (US); Ethan LEVIN, San Francisco, CA (US)

(21) Appl. No.: 15/323,936

(22) PCT Filed: Oct. 3, 2014
(86) PCT No.: PCT/US2014/058957

§ 371 Date: Jan. 4, 2017

Related U.S. Application Data
(60) Provisional application No. 61/886,805, filed on Oct. 4, 2013.

(51) Int. Cl. A61N 5/06

(52) U.S. Cl. CPC A61N 5/0616; A61N 5/062; A61N 2005/0627; A61N 2005/0661; A61N 2005/067

(57) ABSTRACT

A method for testing diseased skin for treatment of the diseased skin, comprising the steps of administering a plurality of increasing doses of phototherapy directly to regions of an area of diseased skin and analyzing the area of the diseased skin to assess the doses at which burning and blistering of the diseased skin occurs, determining a maximum dose of phototherapy that can be administered to the diseased skin based on the assessment of the doses at which the burning and the blistering of the diseased skin occurs, and treating the diseased skin.

### FIELD OF THE INVENTION

The present invention relates generally to treatment of skin disorders and more particularly to a method of treatment protocols for skin disorders.

### BACKGROUND OF THE INVENTION

Skin disorders including atopic dermatitis, dyshidrosis, eczema, litchen planus, psoriasis, and vitiligo are conditions that commonly affect millions of people. Skin disorders can range from mild to severe and can lead to substantial morbidity, can cause psychological stress, and can have a profound negative impact on a patient's quality of life.

For example, psoriasis, is a chronic condition that affects approximately about 1% to about 3% of the population with many new cases being diagnosed each year. Symptoms can range from minor drying and/or flaking of small patches of skin to severe cases with drying and/or flaking affecting large areas of an individual's body.

There are various types of psoriasis a person can be diagnosed with depending on the affected area of the person's body and/or their symptoms. Plaque psoriasis (e.g., psoriasis vulgaris), which accounts for about 80% to about 90% of patients, typically appears as red blotches or patches with dry, silvery scales. Guttate psoriasis appears as numerous small round spots. Flexural psoriasis (inverse psoriasis) can typically be found in skin folds and appears as smooth inflamed patches of skin. Pustular psoriasis appears as raised bumps. Erythrodermic psoriasis usually causes with severe itching, swelling, and pain that may involve the widespread inflammation and exfoliation of the skin. Fingernails and toenails may be affected by nail psoriasis, and often undergo a variety of changes in the appearance that can include small indentations in the nails (e.g., pitting), lifting, discoloration, thickening, and crumbling.

The severity of psoriasis can be classified or “scored” in a variety of ways. In a classification method that is based on the surface area of tissue affected, psoriasis can be graded as mild (e.g., affecting less than about 3% of the total area of the body surface (BSA)), moderate (e.g., affecting about 3% to about 10% BSA), or severe (e.g., affecting more than about 10% BSA). To put the percentages into perspective, the palm of a person's hand is about 1% BSA. Other scales may also be employed for measuring the severity of psoriasis. For example, in addition to the size of affected or influenced BSA, factors such as the condition duration, the frequency of disease recurrence, disease activity (e.g., degree of plaque redness, thickness, and scaling), response to previous therapies, and the impact of the disease on the person may also be considered to determine the severity of the disease.

A subject having less than 3% BSA affected by the condition may be considered to have moderate or severe psoriasis if the affected area is accompanied by radical symptoms such as swelling or pain. A subject having a psoriasis condition that is resistant or recalcitrant to one or more known treatments may also be considered to have severe psoriasis regardless of the size of influenced area. Therefore, psoriasis may be characterized as severe if at least one of the following is observed: the area of influenced tissue is greater than about 10% BSA; the condition (e.g., accompanied by pain and/or swelling) persists for a month or more; the disease activity is substantially active; and the disease is resistant to one or more of known treatments. Psoriasis may also be considered severe if the diseased area comprises between about 10% and about 20% BSA of the subject, between about 20% and/or about 30% BSA, or greater than about 30% BSA.

Severity of psoriasis may also be determined according to standard clinical definitions. For example, the Psoriasis Area and Severity Index (PASI) assess psoriasis disease intensity based on the quantitative assessment of three typical signs of psoriatic lesions: erythema, infiltration, and desquamation, combined with the skin surface area involvement in the head, trunk, upper extremities, and lower extremities. PASI scores range from 0 (no disease) to 72 (maximum disease), in which higher scores indicate greater disease severity. Improvements in psoriasis are indicated, for example, as “PASI 50” (50% improvement in PASI from baseline) (e.g., “PASI 90” is a 90% improvement in PASI from baseline).

The Physicians Global Assessment (PGA) also assesses psoriasis activity and clinical response to treatment. PGA is a six-point score that summarizes the overall quality (erythema, scaling, and thickness) and extent of plaques relative to the baseline assessment. A patient's response is rated as worse (negative clearance (disease became worse)), poor (0-24% clearance), fair (25-49% clearance), good (50-74% clearance), excellent (75-99% clearance), or cleared (100% clearance).

Other measures of improvement in the disease state of a psoriasis patient may include clinical responses such as the Dermatology Life Quality Index (DLQI), the Short Form 36 Health Survey (SF-36), and the European Quality of Life-5 Dimensions (EQ-5D).

A common treatment modality for patients with psoriasis is phototherapy where patients are exposed to doses of narrowband (NB) or broadband (BB) ultraviolet light B (UVB) or psoralen plus doses of ultraviolet light A (PUVA) because ultraviolet light is known to improve the symptoms of psoriasis through immunomodulatory mechanisms. However, patients' tolerances for dosage levels of phototherapy can vary greatly.

With conventional UVB phototherapy, UVB dosing is predicated on either a patient's Fitzpatrick Skin Type (i.e., skin color and darkness) or on the measurement of a patient's minimum erythemal dose (MED). A patient's MED is the dose of UVB that generates a significant red erythemal skin response in normal/healthy tissue. However, neither of these two methods of determining a patient appropriate dosing protocol is therapeutically optimal and typically results in dosing at levels that are very conservative that in turn results in a reduced therapeutic benefit. This is because the Fitzpatrick Skin Type is merely a guess at a patient's maximum tolerable dose (MTD) (based on historical norms that do not apply to many patients) and the fundamental limitations of the MED only measures the tolerance of healthy/normal tissue, not the diseased tissue being treated. It either case, many patients are regularly administered sub-optimal UVB dosing when clinicians, recognizing that current dosing paradigms are only a crude guess, initiate dosing at even lower levels than might be expected. They do so to avoid unintentional dosing at higher levels than the MED that might be above a patient's minimal blistering dose (MBD) leading to extreme erythema and extreme blistering. This problem is exasperated by the fact that the optimum dose, i.e., MTD, a dose near, but just lower than the MBD, can vary greatly for each individual, making it very difficult, if not impossible to generalize a patient's optimum dose. As such, the lack of having an objective way to determine a patient's MBD prevents clinicians from dosing more effectively at a patient's optimum dose level, which could significantly lower the total number of required treatment sessions.

As a result of the typically high number of treatments sessions required because in many cases of conservative treatment, the use of phototherapy is typically limited by the overall inconvenience. Poor compliance with the necessary regimen of regular treatment sessions results because of the time, travel, and the cost required, in many cases, to effectively treat the disease. Other less effective therapies (e.g., topical prescriptions and over-the-counter topical creams) are often the patient's more convenient fallback option.

### SUMMARY OF THE INVENTION

The present invention relates generally to a method of testing and subsequent treatment of a subject (human and non-human) having a skin disease in order to determine the optimal and maximally efficacious dosing of phototherapy.

In an embodiment, a method for treating a skin disease comprises the steps of administering a plurality of doses of phototherapy at increasing intervals to area of diseased skin, analyzing the area of the diseased skin and assessing the doses at which burning and blistering of the diseased skin occurs, determining a maximum interval of phototherapy that can be administered to the diseased skin and treating the diseased skin below or at the maximum dose. In an embodiment the method of treatment is directed at treating psoriasis. In an embodiment, the skin disease can be treated at approximately about at at least one of at a minimal blistering dose, at one or two levels below a minimal blistering dose or at about a minimal erythema dose.

### BRIEF DESCRIPTION OF THE DRAWINGS

FIG. 1A illustrates testing of a plurality of spots with various dosage levels;

FIG. 1B illustrates an embodiment of dosage interval levels for the plurality of spots in FIG. 1A;

FIG. 2 shows the effect of a dose of phototherapy applied directly to an area of diseased skin at which the skin reacts with minimal erythema (i.e., Minimal Erythema Dose);

FIG. 3 shows the effect of a dose of phototherapy applied directly to an area of diseased skin at which blistering occurs (i.e., Minimum Blistering Dose); and

FIG. 4 shows the dose at which a patient's skin reacts with minimal erythema and the dose at which blistering occurs.

### DETAILED DESCRIPTION OF EMBODIMENTS OF THE INVENTION

As discussed above, known phototherapy treatment of skin disorders (e.g., psoriasis) involves testing non-affected areas of a patient's skin tissue by applying doses of varying levels of UV light in an attempt to determine the patient's maximum tolerable dose. However, diseased skin, has a much higher tolerance to UV light than non-affected skin. Thus, testing non-affected areas of skin does not provide an accurate assessment of the patient's tolerance and in turn maximum dosage that can be applied to a region of the patient's skin that is affected by a skin disorder.

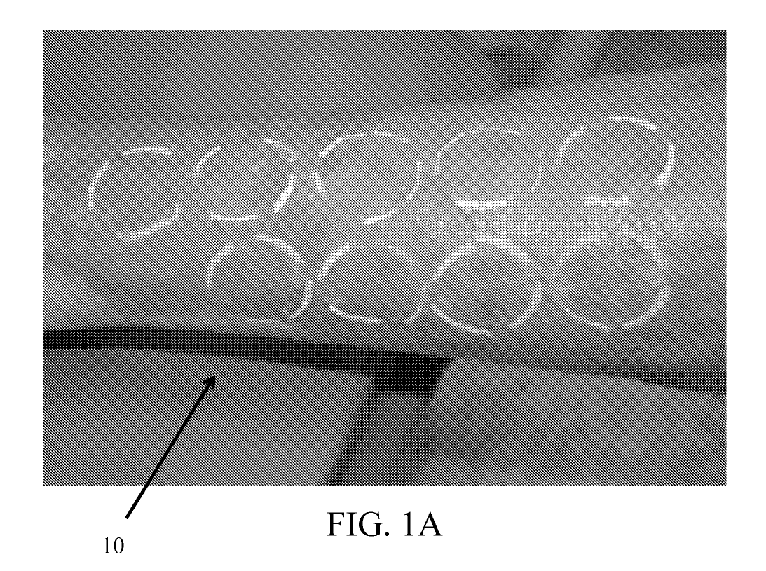

FIGS. 1-3 illustrate an embodiment of a method of testing a region of skin affected by a skin disorder prior to phototherapy treatment by applying dosages of incremental wavelengths of UV light directly to the region of skin affected by the skin disorder that is designated hereinafter by reference numeral 10. In an embodiment, the skin disorder is psoriasis.

UV phototherapy utilizes light in the UVB band, which extends in wavelength between about 280 nanometers and about 320 nanometers. Psoriasis-afflicted tissue can be effectively rehabilitated with light having wavelengths between about 300 nanometers and about 310 nanometers. Light having a wavelength spectrum between about 295 nanometers and about 325 nanometers can be effective in healing the tissue as well. However, due to the intensity of the light applied to a patient, there is a risk of some undesirable side effects of phototherapy to treat psoriasis and other skin conditions such as DNA damage (e.g., skin cancer). Therefore, to treat diseased skin and joints of a subject under conditions that can maximize a likelihood of placing the diseased tissue into remission while minimizing the risk of erythema and/or DNA damage, a physician will typically treat a patient in a range of between about 295 nanometers and about 320 nanometers, more specifically, between about 300 nanometers and about 310 nanometers, and even more specifically at about 308 nanometers.

The fluence of light having wavelengths distributed between about 300 and about 310 nanometers has been determined to range between about 500 mJ/cm² and about 5000 mJ/cm². More specifically, the fluences preferably range between about 100 mJ/cm² and about 8 J/cm², and between about 300 mJ/cm cm² and about 3 J/cm cm². Other dosages are also possible depending upon where blistering of the plaque takes place. To determine such blistering the laser radiation may be applied in increments of typically 50, 100 or 200 mJ/cm². In an embodiment, one or two such treatments can provide significant improvement of the afflicted area of skin as opposed to 20 treatments by prior methodology.

In embodiments, phototherapy comprises an average power of between about 0.3 watts and about 0.5 watts, between about 2 watts and about 3 watts, between about 2.5 watts and about 4.5 watts, or between about 4.8 watts and about 7.2 watts. Other average powers are also possible (e.g., 10 watts or more). In embodiments, phototherapy comprises energy between about 8 mJ/pulse and about 15 mJ/pulse or between about 12 mJ/pulse and about 18 mJ/pulse. Other pulse energies are also possible based on the power of the laser and the exposure area.

In embodiments, phototherapy comprises a repetition rate of between about 50 pulses/second (Hz) and about 100 Hz (e.g., about 92 Hz), between about 100 Hz and about 500 Hz. In embodiments, the repetition rate is between about 125 Hz and about 175 Hz (e.g., about 154 Hz), between about 150 Hz and about 300 Hz (e.g., about 200 Hz, about 225 Hz, about 250 Hz), or between about 350 Hz and about 450 Hz (e.g., about 400 Hz). In an embodiment, other repetition rates are possible and higher repetition rates may reduce the treatment duration, for example, because the phototherapy device may able to deliver the treatment dosage in a shorter period of time depending on the power per pulse.

As shown in FIGS. 1A and 1B, a region of an appendage affected by psoriatic plaque is marked with a plurality of circles for pre-treatment phototherapy testing. In an embodiment, nine regions are tested at increasing intervals. However, the number of regions of diseased tissue tested can vary, for example, depending on the size of the affected region of diseased tissue or previously known treatment tolerances. After marking the regions of diseased tissue, a laser can be used to “serial test” the marked regions by apply increasing doses of UV light in the marked regions to the diseased tissue (see FIG. 1B). After applying varying levels of UV light to the psoriatic plaque diseased area, the patient is then typically sent home for about 24 to 48 hours. After 24 to 48 hours, the patient then returns to the treatment center where the area of diseased skin tissue is observed to assess the dosage level at which burning (MED) 12 and/or blistering (MBD) 14 of the diseased skin tissue begins to occur.

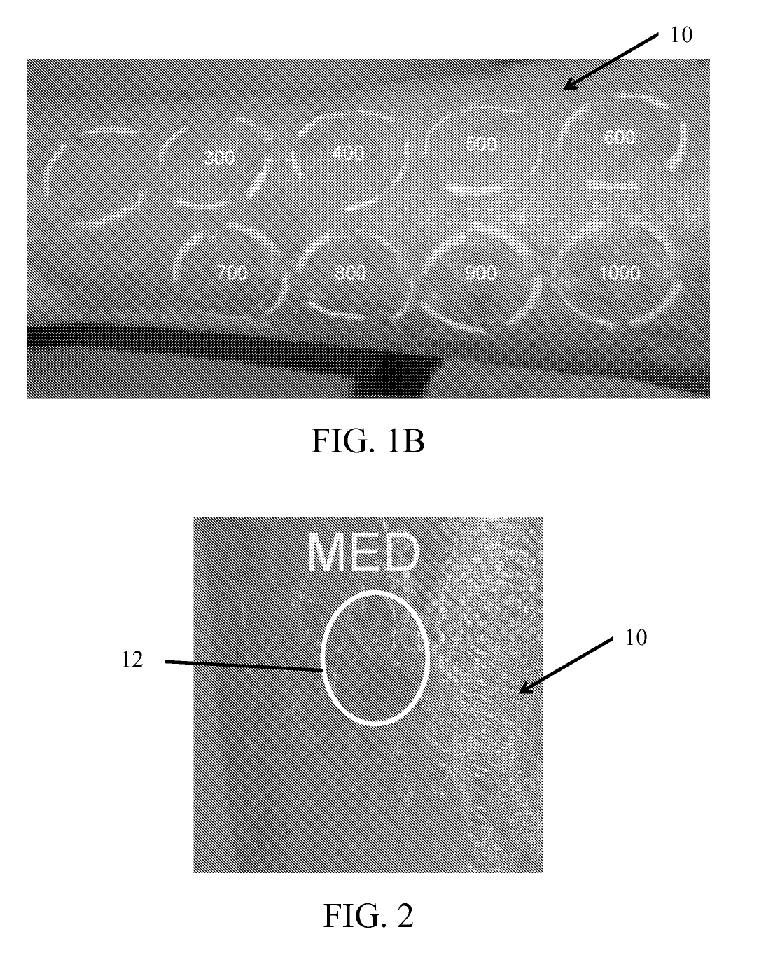

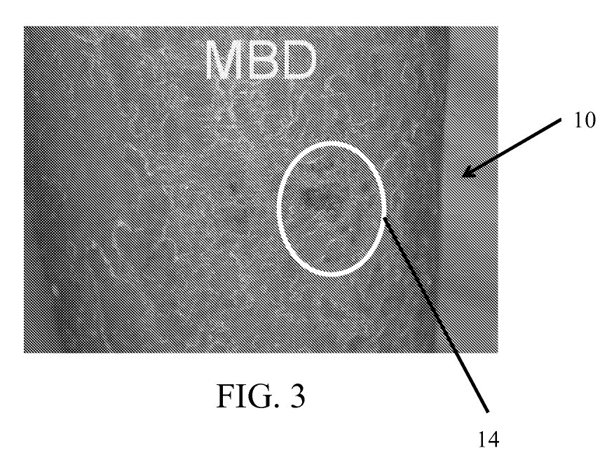

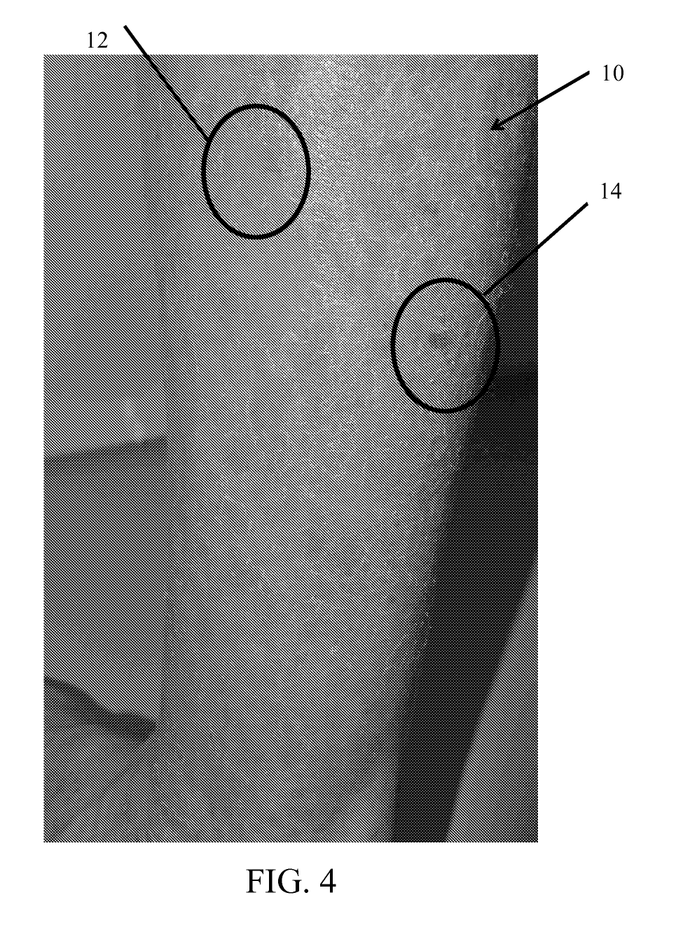

By observing the MED 12 as shown in the appendage in FIGS. 2 and 4 where a region of reddened skin is present and the MBD 14, as shown in FIGS. 3 and 4, where the treated region of diseased skin begins to blister, the “upper limit” dose that can be used to treat the patient can be determined. Based on the MBD, depending on the patient's tolerance and the severity of diseased tissue, among other factors, the patient can be treated at the MBD, just below the MBD (e.g., one or two dosage levels below the MBD), or closer to the MED of the diseased skin tissue.

Exposing a psoriatic area to high doses of UV light will cause faster clearing and place the skin condition into remission much faster and for a longer period of time than a lower dosage of UV light. As such, by directly testing diseased skin tissue to assess the optimal dose of laser treatment of psoriasis based on MBD allows for treatment to be close to the most aggressive and, therefore, more efficacious, but still well-tolerable dosage.

By treating a patient at or near their MBD, the number of treatment sessions required to place the diseased tissue into remission can be greatly reduced, and in an embodiment, such reduction in the number of treatments may achieve the desired result and yet decrease the total quantity or cumulative deposition of UVB light to which skin is exposed.

The risk of erythema, skin cancer and skin damage generally associated with premature aging depends the total number of photons or amount of UVB radiation directed on the skin. As such, raising the dosage in a single treatment, a couple treatments, or a few treatments can reduce the number of treatment sessions and result in a lower overall number of absorbed photons or UVB radiation, thereby reducing the risk of cancer and other skin damage. A fewer number treatments can also result in a significant reduction in hyperpigmentation that would normally be experienced due to the body's sun tanning response after each treatment. Additionally, lower numbers of treatments may also provide a higher degree of compliance of a subject to an otherwise difficult regimen involving a significant number of visits to the physician.

To treat the diseased skin, after assessing a patient's tolerance, in an embodiment, ultraviolet light is delivered to each affected region of the body, for example, by an excimer laser, as described in U.S. Pat. Nos. 7,144,248 and 7,276,059, each herein incorporated by reference in their entirety. In an embodiment, UV lamps, intense pulsed light (“IPL”) devices, light-emitting diode (“LED”) devices (e.g., available from Photo Therapeutics, Ltd. of Altrincham, United Kingdom and Photo Therapeutics, Inc. of Carlsbad, Calif.), or other phototherapy devices that are known or will be developed in the future can be employed to generate the UV light.

In an embodiment, ultraviolet light is directed only onto the affected regions. In an embodiment, ultraviolet light is directed onto the lesional as well as surrounding paralesional tissue, which although appearing normal is diseased tissue.

In an embodiment, phototherapy treatment of diseased psoriatic plaque can be combined with the use of a topical spray and/or ointment, such as clobetasol spray and calcitriol ointment to minimize phototoxicity. The use of a topical spray and/or ointment is typically used for the treatment of moderate-to-severe generalized psoriasis.

In some embodiments, phototherapy is administered or received without any help from a light-sensitizing agent. Alternatively, in an embodiment, light-sensitizing agents may be used, for example to increase the sensitivity of a cell to UV. In certain such embodiments, one or more light-sensitizing agents may be applied to the subject or received by the subject before or after phototherapy. Examples of light-sensitizing agents include, but are not limited to, coal tar, psoralen, acitretin, and salicylic acid. By avoiding treatment of unaffected portions of skin, the dosage can be raised well above conventional dosages as the affected areas will tolerate substantially higher doses without increased risk of side effects.

In an embodiment, an excimer laser can be used to generate short high power pulses of light having a wavelength of about 308 nanometers. These pulses can be high in peak power, e.g., about half a million watts, but short in duration, for example, lasting much less than about 100 nanoseconds (e.g., about 30 nanoseconds). The laser, however, may produce a plurality of such pulses at a repetition rate of about 100, 150, 200, 250, 300, 400, 450, or 500 Hz, and ranges therebetween. Tissue exposed to a plurality of these short pulses will increase in temperature slightly with application of each pulse. The cumulative effect of the plurality of pulses to raise the temperature of the tissue to a certain amount depends in part on the heat capacity of the tissue. The energy from the laser may be spread over a long enough period of time so as to permit sufficient dissipation to avoid excessive build-up of heat from the plurality of short pulses. Thermal damage caused by raising the temperature of the skin above, for example, the blister temperature of 50° C., can thereby be reduced, mitigated, or prevented. The duration of exposure of the affected tissue to the therapeutic doses of UV light, however, depends on the particular dose level.

In an embodiment, high doses of UV illumination are directed to an area of skin that is between about 25 cm² and about 6,000 cm², between about 25 cm² and about 5,000 cm², between about 25 cm² and about 4,000 cm², between about 25 cm² and about 3,000 cm², between about 25 cm² and about 2,000 cm², between about 25 cm² and about 1,800 cm², between about 25 cm² and about 1,600 cm², between about 25 cm² and about 1,400 cm², between about 25 cm² and about 1,200 cm², between about 25 cm² and about 1,000 cm², between about 25 cm² and about 800 cm², between about 25 cm² and about 600 cm², between about 25 cm² and about 500 cm², between about 25 cm² and about 400 cm², between about 25 cm² and about 300 cm², between about 25 cm² and about 200 cm², between about 25 cm² and about 100 cm², between about 25 cm² and about 50 cm², between about 1 cm² and about 25 cm², between about 2,000 cm² and about 5,000 cm², between about 3,000 cm² and about 5,000 cm², between about 4,000 cm² and about 5,000 cm², between about 2,000 cm² and about 6,000 cm², between about 3,000 cm² and about 6,000 cm², between about 4,000 cm² and about 6,000 cm², between about 5,000 cm² and about 6,000 cm², or ranges included therein.

In some embodiments, high doses of UV illumination are directed to an area on the skin that is less than about 6,000 cm², less than about 5,000 cm², less than about 4,000 cm², less than about 3,000 cm², less than about 2,000 cm², less than about 1,800 cm², less than about 1,600 cm², less than about 1,400 cm², less than about 1,200 cm², less than about 1,000 cm², less than about 800 cm², less than about 600 cm², less than about 500 cm², less than about 400 cm², less than about 300 cm², less than about 200 cm², less than about 100 cm², less than about 50 cm², less than about 25 cm², or values therebetween.

In some embodiments, the high doses of UV illumination are directed to an area on the skin that is between about 1% BSA and about 30% BSA, between about 1% BSA and about 25% BSA, between about 1% BSA and about 20% BSA, between about 1% BSA and about 15% BSA, between about 1% BSA and about 10% BSA, between about 1% BSA and about 5% BSA, between about 3% BSA and about 30% BSA, between about 3% BSA and about 25% BSA, between about 3% BSA and about 20% BSA, between about 3% BSA and about 15% BSA, between about 3% BSA and about 10% BSA, between about 3% BSA and about 5% BSA, between about 10% BSA and about 30% BSA, between about 10% BSA and about 25% BSA, between about 10% BSA and about 20% BSA, between about 10% BSA and about 15% BSA, or ranges included therein. In some embodiments, the high doses of UV illumination are directed to an area on the skin that is less than about 30% BSA, less than about 25% BSA, less than about 20% BSA, less than about 15% BSA, less than about 10% BSA, less than about 5% BSA, less than about 3% BSA, less than about 1% BSA, or values therebetween.

Although the description above and accompanying drawings contains much specificity, the details provided should not be construed as limiting the scope of the embodiments, but merely as describing some of the features of the embodiments. The description and figures should not to be taken as restrictive and are understood as broad and general teachings in accordance with the present invention. While the embodiments have been described using specific terms, such description is for illustrative purposes only, and it is to be understood that modifications and variations to such embodiments, including, but not limited to, the substitutions of equivalent features and terminology may be readily apparent to those of skill in the art based upon this disclosure without departing from the spirit and scope of the invention.

1. A method for treating a skin disease, comprising the following steps:
administering a plurality of doses of phototherapy at increasing intervals to area of diseased skin;
analyzing the area of the diseased skin and assessing the doses at which burning and blistering of the diseased skin occurs;
determining a maximum dose of phototherapy that can be administered to the diseased skin based on the assessment of the doses at which the burning and the blistering of the diseased skin occurs; and
treating the diseased skin below or at the maximum dose.

2. The method of claim 1, wherein the diseased skin is psoriasis.

3. The method of claim 1, further comprising the step of treating the diseased skin at approximately about at least one of at a minimal blistering dose, at one or two levels below a minimal blistering dose or at about a minimal erythema dose.

4. The method of claim 1, further comprising the step of marking the area of diseased skin prior to administering the plurality of doses of phototherapy and then administering the doses of phototherapy within the markings.

5. The method of claim 1, wherein eight doses of increasing phototherapy are administered to eight region of diseased skin.

6. The method of claim 1, wherein nine doses of increasing phototherapy are administered to nine region of diseased skin.

7. The method of claim 1, further comprising the step of determining the minimal erythema dose applied to the area of the diseased skin after the step of administering the plurality of doses of phototherapy.

8. The method of claim 1, further comprising the step of determining the minimal blistering dose applied to the patch of diseased skin after the step of administering the plurality of doses of phototherapy.

9. The method of claim 1, further comprising the step of waiting about 24 to 48 hours after administering the plurality of doses of phototherapy and then analyzing and assessing the area of diseased skin to determine an upper dosing limit that should be used to treat the patient.

10. The method of claim 1, further comprising the step of applying a light-sensitizing agent to the area of diseased skin.

11. The method of claim 1, further comprising the step of applying a topical spray or ointment to the area of the diseased skin to minimize phototoxicity.

12. The method of claim 1, further comprising the step of treating the diseased skin by applying at most three doses of phototherapy to place the diseased skin into remission.
